(19) United States
(12) Patent Application Publication
(10) Pub. No.: US 2008/0047835 A1
(43) Pub. Date: Feb. 28, 2008

(54) Genomic DNA Purifier

(76) Inventor: William P. MacConnell, Encinitas, CA (US)

Correspondence Address: NYDEGGER & ASSOCIATES, 348 OLIVE STREET, SAN DIEGO, CA, 92103, US

(21) Appl. No.: 11/466,398

(22) Filed: Aug. 22, 2006

(51) Int. Cl. C07K 1/26; G01N 27/00

(52) U.S. Cl. 204/456; 204/606

(57) ABSTRACT

A system and method for purifying genomic DNA requires the use of a cassette that is formed with a plurality of wells. Each well has first and second apertures that are respectively covered by a section made of an electrophoretic filter medium (e.g. agarose). In use, a lysate is loaded into the well while the cassette is submerged in a buffer fluid. A voltage cycle is then applied to alternate between forward and reverse electrophoresis in the well, to separate impurities from the lysate for purification of genomic DNA.

### FIELD OF THE INVENTION

The present invention pertains generally to systems and methods for purifying genomic DNA. More particularly, the present invention pertains to systems and methods that rely on an electrophoretic process for removing impurities from a lysate to purify genomic DNA. The present invention is particularly, but not exclusively, useful as a system or method for purifying genomic DNA by cycling a lysate through a sequence of forward and reverse (backward) electrophoretic processes.

### BACKGROUND OF THE INVENTION

By definition, lysis refers to the disintegration of a cell by a rupture of the cell wall or membrane. A lysate is the result of this disintegration. As is widely recognized by biologists, a lysate can be used for many different purposes. In particular, it happens that genomic DNA can be recovered from a lysate and subsequently used in molecular biology and diagnostic procedures such as amplification using the polymerase chain reaction (PCR) and in other important applications, such as forensics, medicine and genetic research. Not surprisingly however, before it can be effectively used as a template for the above purposes, the genomic DNA needs to be purified. This requires removing impurities from the lysate containing the target genomic DNA.

It is well known that genomic DNA can be recovered from blood, dissected tissue, or bacteria (both gram positive and gram negative), as well as other sources. When samples of these materials are lysed, however, the process will invariably yield numerous impurities in addition to the genomic DNA. For almost all applications, it is desirable that as many impurities as possible be removed from the lysate, before any further processing. Typically, these impurities will include molecules such as digested RNA, protein, detergent, lipids, and cellular debris. In general, the molecules of these impurities are smaller than the genomic DNA.

Empirical data indicates that the smaller molecules in a lysate (i.e. the impurities) can be effectively filtered from the genomic DNA that is in the lysate. In particular, it is known that such filtering can be caused when the lysate, as a solution, is electrophoretically driven into contact with an electrophoretic filter medium (such as an agarose). Interestingly, during initial contact of the lysate with an agarose, it has been noticed that the genomic DNA is hindered from entering the agarose for a substantial period of time. On the other hand, the smaller impurities are not so hindered from entering or passing through the agarose.

In light of the above, it is an object of the present invention to provide a system and method for purifying genomic DNA that incorporates a cycle of reverse (backward) and forward electrophoresis to remove impurities from a lysate containing genomic DNA. Another object of the present invention is to provide a system and method that can accomplish the purification of genomic DNA without employing machine moveable components. Still another object of the present invention is to provide a system and method for purifying genomic DNA that is easy to use, simple to implement and cost effective.

### SUMMARY OF THE INVENTION

In accordance with the present invention, a system and method for purifying genomic DNA involves a cassette that is formed with a plurality of hollow, rectangular channels. Importantly, the channels are oriented substantially parallel to each other. Also incorporated into the cassette are two sections of an electrophoretic filter medium, such as agarose. As recognized by the present invention, this electrophoretic filter medium will typically be an agarose. It is possible, however, that another medium, such as a solution of polymerized acrylimide, could be used as an alternative. For purposes of disclosure, the term “agarose section” will be used to indicate such a structure. In any event, two such agarose sections are positioned in each channel and distanced from each other. As so positioned, the agarose sections establish a well in their respective channel that is located between the sections. Preferably, for purposes of the present invention, the agarose sections are formed as a gel cast and are made of approximately 1% agarose. Additionally, each channel is formed with an opening for access into the well. In combination, the parallel channels with their respective openings, and the agarose sections with the well between them, create the cassette. Since the channels and the well are separated from adjacent channels and wells by plastic walls, samples do not intermix during the purification process and, thus, they do not cross-contaminate.

Along with the cassette, the system of the present invention requires an electrophoresis rig. Structurally, the rig has opposite first and second ends, with a pair of parallel, same-length side walls extending therebetween. This creates a basin for holding a buffer fluid into which the cassette can be submerged for operation of the system. Further, respective electrodes are mounted at the opposite ends of the rig, and a voltage source is connected to the electrodes to generate an electric field in the basin that is directed between the two electrodes. Also, a fluid pump is provided to pump the buffer fluid into, and out of, the basin.

In the operation of the present invention, a lysate containing genomic DNA is loaded into the wells of the cassette. This loading is done through the respective channel openings. The loaded cassette is then positioned in the basin of the electrophoresis rig and submerged in the buffer fluid. With the cassette positioned in the rig, the voltage source can then be activated to create an electric field for electrophoresis of the lysate. As intended for the present invention, electrophoresis is done in parallel, and within in each respective well in accordance with a timed program cycle. Importantly, the timed program cycle includes both a forward and a reverse electrophoresis of the lysate in each well.

As implied above, the process of the present invention can simultaneously accomplish the genomic DNA purification of a plurality of samples. Depending on the size of the lanes in the cassette (i.e. the agarose sections and the well between them), and the number of lanes in the cassette, a large number of samples of varying size can be accommodated. Specifically, in addition to the number of different samples that are to be processed, the sample size can vary from rather large to very minute. In any event, the purification of many samples can be accomplished simultaneously.

As envisioned for the present invention, the timed program cycle for electrophoresis preferably includes an initial forward electrophoresis wherein 100 volts DC are applied for about 10 minutes. The direction of the electric field is then changed for a reverse electrophoresis with 100 volts DC applied for about 2 minutes. This, in turn, is followed by another forward electrophoresis with 150 volts DC applied for around 7 minutes. And finally, there is another reverse electrophoresis with 150 volts DC applied for approximately 2 minutes. During this timed program cycle, impurities in the lysate are separated from the genomic DNA for purification of the genomic DNA. As will be appreciated by the skilled artisan, many variations on the backward and forward cycle of the electrophoretic program are envisioned for use with the present invention. For example, cycle changes can result from changes in the magnitude of the voltage that is used to create the electric field. Cycle changes can also result when the time duration in which the electric field is created is varied. Also, the fluid pump can be operated during a cycle or thereafter to renew the buffer fluid for each timed program cycle. At the end of a timed program cycle, the cassette can be removed from the electrophoresis rig and the purified genomic DNA can be removed from the wells of the cassette through the channel openings.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an exploded perspective view of a system for purifying genomic DNA in accordance with the present invention;

FIG. 2A is a cross sectional view of a cassette unit as seen along the line 2-2 in FIG. 1 during an electrophoretic process; and

FIG. 2B is a view as in FIG. 2A during electrophoresis in an opposite direction.

### DESCRIPTION OF THE PREFERRED EMBODIMENTS

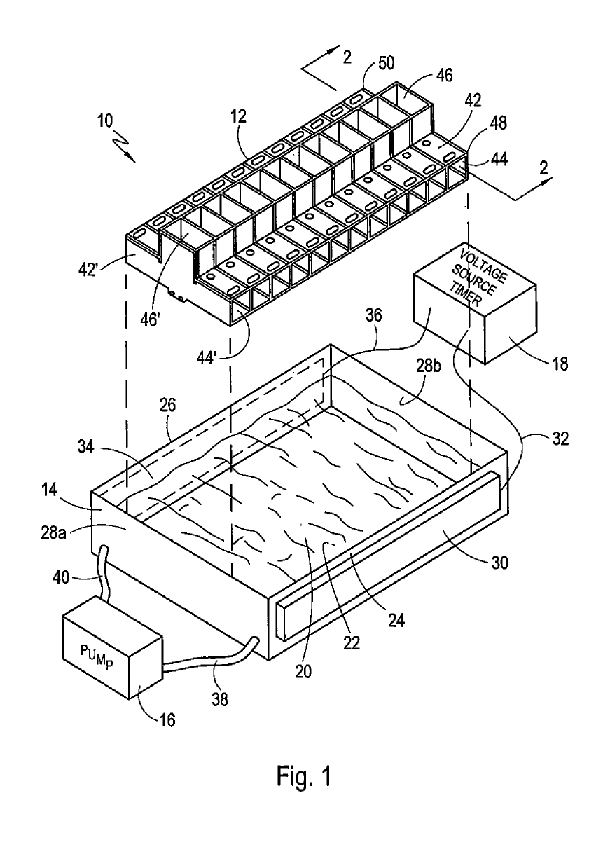

Referring initially to FIG. 1, a system for purifying genomic DNA in accordance with the present invention is shown, and is generally designated 10. As shown, the system 10 includes a cassette 12 and an electrophoresis rig 14 that is dimensioned for receiving the cassette 12. Further, the system 10 includes a pump 16 that is connected for buffer fluid communication with the rig 14, and it also includes a voltage source/timer 18 that is electrically connected with the rig 14.

In more detail, and still referring to FIG. 1, the rig 14 is shown formed with a basin 20 for holding a buffer fluid 22. Structurally, the basin 20 is bounded by the combination of a first end 24, an opposite second end 26, and a pair of opposed sidewalls 28a, 28b that extend in parallel between the ends 24 and 26. In FIG. 1 it is also shown that an electrode 30 is mounted on the first end 24, and that this electrode 30 is electrically connected via line 32 with the voltage source/timer 18. Similarly, an electrode 34 is mounted on the second end 26. The electrode 34 is also electrically connected with the voltage source/time 18, but via a line 36. FIG. 1 also shows that the pump 16 is connected for buffer fluid communication with the basin 20 via tubes 38 and 40.

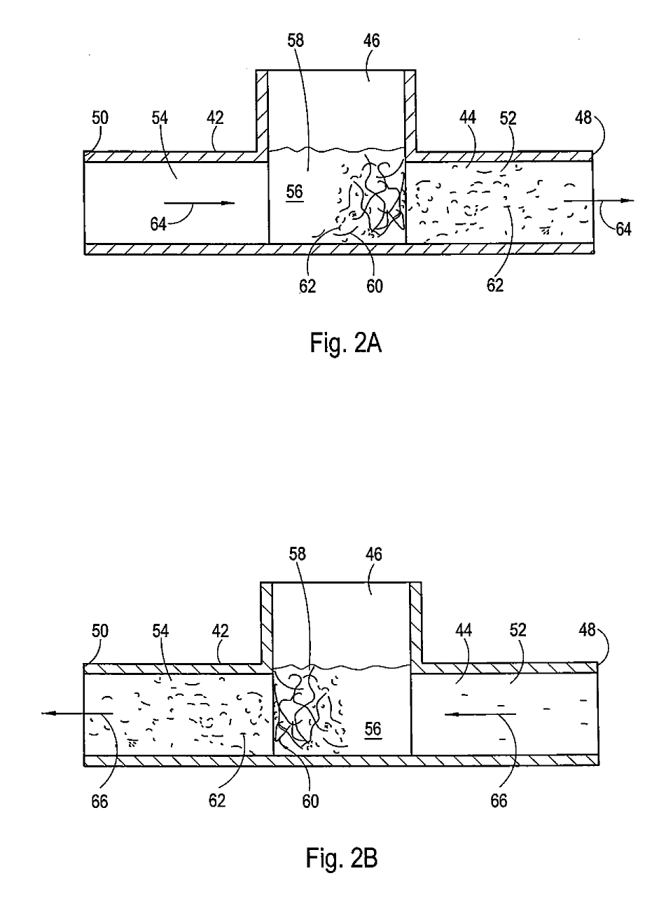

The construction of the cassette 12 will, perhaps, be best appreciated by cross-referencing FIG. 1 with FIG. 2A. First, with reference to FIG. 1, it can be appreciated that the cassette 12 includes a plurality of individual units 42. For simplicity, however, only the units 42 and 42′ at the opposed ends of the cassette 12 have been actually labeled. Nevertheless, as envisioned for the present invention, all units 42 are substantially identical. Thus, when cross-referencing FIG. 1 with FIG. 2A, it will be appreciated that each unit 42 includes an elongated, substantially rectangular channel (lane) 44, and that each unit 42 has a respective opening 46 which is located midway between the ends 48 and 50 of the channel 44. As will be appreciated by the skilled artisan, more or fewer units 42, than shown in FIG. 1, can be incorporated into a cassette 12.

With reference now to FIG. 2A, it is to be appreciated that the disclosure given here for the unit 42 is exemplary of all units 42 in the cassette 12. With this in mind, FIG. 2A shows that the unit 42 includes an agarose section 52 and an agarose section 54. Specifically, the agarose sections 52 and 54 are located in the channel 44 with a gap between them that establishes a well 56. As shown, access to the well 56 can be had through the opening 46. As envisioned for the system 10 of the present invention, the agarose sections 52 and 54 extend through the channel 44 from the well 56 to the respective ends 48 and 50. Stated differently, in addition to the opening 46, the well 56 is formed with apertures that are located between the well 56 and the channel 44. In line with the above disclosure, these apertures between well 56 and channel 44 are respectively covered by the agarose sections 52 and 54. Regardless how characterized, both agarose sections 52 and 54 are each, preferably, a gel cast that is made of agarose solidified in a buffer solution. As mentioned above, instead of agarose, a solution of polymerized acrylimide may be used. Indeed, any electrophoretic filter medium of a type well known in the pertinent art can be used for the sections 52 and 54. The buffer fluid is typically comprised of 20 mM Tris-Acetate PH 8.0 with 0.062 mM Na₂ EDTA.

### Operation

In the operation of the system 10 of the present invention, a lysate 58 is prepared, and is introduced into the well 56 of the unit 42. Typically, this lysate 58 will include genomic DNA 60, as well as various impurities 62. The actual lysate 58 that is prepared for purification by the system 10, however, will depend on several factors. By way of example, a lysate 58 can be prepared by mixing a sample material (e.g. blood, tissue, crude buffy coat fraction of blood, or bacteria) with 0.1% SDS, 0.5 μg/ul proteinase K, 0.01 μg/ml Rnase, and 2 mM EDTA. This will create a mixture that is then heated to a temperature in the range between 55° C. and 70° C. The temperature is then held overnight (e.g. up to approximately ten hours), depending on the starting sample type. The resultant lysate 58 can then be pipetted into the well 56.

Once the cassette 12 is loaded (i.e. the wells 56 are filled with lysate 58), the cassette 12 is positioned in the basin 20 of the electrophoresis rig 14. Specifically, this is accomplished by locating the ends 48 of the units 42 adjacent the first end 24 (i.e. electrode 30), and locating the ends 50 adjacent the second end 26 (i.e. electrode 34). Essentially, this orients the channels 44 so that they extend directly between the electrodes 30 and 34. At this point, if not previously done, the basin 20 can be filled with buffer fluid 22. The voltage source/timer 18 can then be activated to conduct a timed program cycle.

For the present invention, a timed program cycle involves the activation of the voltage source/timer 18 to establish an electric field, E for electrophoresis. Specifically, the electric field E for electrophoresis is generated by the electrodes 30 and 34, and is directed through the channels 44 of cassette 12 between the electrodes 30 and 34. Importantly, as envisioned for the system 10, the magnitude and direction of the electric field E is changed in a predetermined manner.

In a typical timed program cycle, the electric field E is generated by the voltage source/timer 18 and is oriented in the direction of arrows 64 (see FIG. 2A). This establishes a forward electrophoresis with 100 volts DC applied by the voltage source/timer 18 for thirty (30) minutes. As illustrated in FIG. 2A, this forward electrophoresis will drive the genomic DNA 60 and impurities 62 in the lysate 58 into contact with the agarose section 52. The agarose section 52 will then filter the impurities 62 out of the lysate 58 by hindering the passage of the genomic DNA 60 through the agarose section 52. After thirty (30) minutes, the voltage source/timer 18 reverses the direction of the electric field E into the direction indicated by arrows 66 in FIG. 2B. The consequent reverse electrophoresis is then performed with 100 volts DC applied for five (5) minutes. During this reverse electrophoresis, the agarose section 54 performs the same function as disclosed above for the agarose section 52. Another forward electrophoresis is then performed (see FIG. 2A) with 100 volts DC applied for thirty (30) minutes. And, this is then followed by another reverse electrophoresis (see FIG. 2B) with 100 volts DC applied for five (5) minutes. The buffer is then changed and another forward-reverse cycle is executed. Specifically, another forward electrophoresis with 100 volts DC is applied for thirty (30) minutes. This time, however, the forward electrophoresis is followed by a reverse electrophoresis with ten (10) volts DC for 5 minutes. Together, the complete sequence of forward-reverse electrophoresis constitutes a timed program cycle. After a timed program cycle has been performed, the now purified genomic DNA 60 can be removed from the well 56 through the opening 46.

In another aspect of the present invention it is to be appreciated that the pump 16 can be operated to provide for a running buffer fluid 22 during a timed program cycle. Further, the pump 16 can be operated after a timed program cycle to renew buffer fluid 22 in the basin 20. Still further, as an alternative, the cassette 12 can be simply removed from the basin 20 while the buffer fluid 22 is replaced.

With the above in mind, it will be appreciated that the system 10 of the present invention is intended to simultaneously process many samples for genomic DNA purification during a single run (cycle). Moreover, the system 10 can employ one or more cassettes 12 during each run. On the other hand, a same run can be used regardless of the number of samples being processed. As also mentioned above, for purposes of purifying samples, the size and number of channels (lanes) 44 can be varied as desired. The versatility of the system 10 is further underscored by the fact the operator can change the electrophoretic cycle, as desired. This is done by changing the magnitude of the voltage that is used to create the electric field E for electrophoresis, or by changing the time duration of portions of the procedure. Also, since the genomic DNA does not actually leave the well 56 during a run, the recovery of the total amount of genomic DNA found in the sample is maximized. By the same token, purifications from samples containing only trace amounts of genomic DNA are possible.

While the particular Genomic DNA Purifier as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for purifying genomic DNA which comprises:
a cassette formed with a plurality of wells for respectively receiving a lysate therein, wherein each said well is formed with a first aperture and with a second aperture;
a first electrophoretic filter medium section and a second electrophoretic filter medium section to respectively cover the first and second apertures of said cassette;
a rig for holding a buffer fluid for submersion of the cassette in the buffer fluid; and
a voltage means mounted on the rig for alternating the direction of an electric field in each well for a respective forward and reverse electrophoresis of the lysate between the first and second apertures, to separate impurities from the lysate for purification of genomic DNA.

2. A system as recited in claim 1 wherein the first section and the second section are each a gel cast made of agarose solidified in buffer solution.

3. A system as recited in claim 1 wherein each well has an opening for introducing the lysate into the well and for removing purified genomic DNA therefrom.

4. A system as recited in claim 1 wherein said rig has a first end and a second end, with a pair of substantially parallel, substantially same-length side walls extending therebetween to create a basin for holding the buffer fluid therein.

5. A system as recited in claim 4 wherein said voltage means comprises:
a first electrode mounted on the first end of said rig; and
a second electrode mounted on the second end of said rig.

6. A system as recited in claim 5 wherein the voltage means further comprises a timing means for performing electrophoresis in a timed program cycle.

7. A system as recited in claim 6 wherein the timed program cycle includes a forward electrophoresis with 100 volts DC applied for 30 minutes and a reverse electrophoresis with 100 volts DC applied for 5 minutes, followed by a forward electrophoresis with 100 volts DC applied for 30 minutes and a reverse electrophoresis with 100 volts DC applied for 5 minutes, followed by a change of buffer fluid, another forward electrophoresis with 100 volts DC applied for 30 minutes and a reverse electrophoresis with 10 volts DC applied for 5 minutes.

8. A system as recited in claim 7 further comprising a pumping means for causing the buffer fluid to flow through the rig.

9. A system as recited in claim 8 wherein the buffer fluid has been renewed after each timed program cycle.

10. A system for purifying genomic DNA which comprises:
an elongated housing having a top wall, a bottom wall and a pair of opposed side walls therebetween, with said housing defining a channel;
a first section made of electrophoretic filter medium positioned in the channel;
a second section made of electrophoretic filter medium positioned in the channel at a distance from said first section to establish a well in the channel therebetween for receiving a lysate therein;
a means for holding the channel in a buffer fluid; and
a voltage means for alternating the direction of an electric field in the well for a respective forward and reverse electrophoresis of the lysate between the first and second sections, to separate impurities from the lysate for purification of genomic DNA.

11. A system as recited in claim 10 wherein the first section and the second section are each a gel cast made of agarose solidified in buffer solution.

12. A system as recited in claim 10 wherein the top wall is formed with an opening for introducing the lysate into the well and for removing purified genomic DNA therefrom.

13. A system as recited in claim 10 wherein the holding means is a rig having a first end and a second end, with a pair of substantially parallel, substantially same-length side walls extending therebetween to create a basin for holding the buffer fluid therein.

14. A system as recited in claim 13 wherein the voltage means comprises:
a first electrode mounted on the first end of the rig;
a second electrode mounted on the second end of the rig; and
a timing means for performing electrophoresis in a timed program cycle.

15. A system as recited in claim 14 wherein the timed program cycle includes a forward electrophoresis with 100 volts DC applied for 30 minutes and a reverse electrophoresis with 100 volts DC applied for 5 minutes, followed by a forward electrophoresis with 100 volts DC applied for 30 minutes and a reverse electrophoresis with 100 volts DC applied for 5 minutes, followed by a change of buffer fluid, another forward electrophoresis with 100 volts DC applied for 30 minutes and a reverse electrophoresis with 10 volts DC applied for 5 minutes.

16. A system as recited in claim 15 further comprising a pumping means for causing the buffer fluid to flow through the rig to renew the buffer fluid after each timed program cycle.

17. A method for purifying genomic DNA using a cassette formed with a plurality of wells, wherein each well is formed with a first aperture and with a second aperture and wherein each aperture is covered by a respective section of electrophoretic filter medium, the method comprising the steps of:
preparing a lysate;
loading each well with the lysate;
submerging the cassette in a buffer fluid; and
alternating the direction of an electric field in each well for a respective forward and reverse electrophoresis of the lysate between the first and second apertures, to separate impurities from the lysate for purification of genomic DNA.

18. A method as recited in claim 17 wherein the preparing step comprises the steps of:
mixing a sample material with proteinase, Rnase, and EDTA to create a mixture; and
heating the mixture at a temperature in the range between room temperature and 70° C. for a duration in the range of 0 minutes to ten hours to effect lysis.

19. A method as recited in claim 18 wherein the alternating step includes the steps of:
alternating between a forward electrophoresis and a reverse electrophoresis; and
subsequently alternating between a forward electrophoresis and a reverse electrophoresis to effect purification.

20. A method as recited in claim 17 further comprising the step of pumping buffer fluid through the rig to renew the buffer fluid after completion of each alternating step.

21. A cassette for use in purifying genomic DNA which comprises:
a plurality of elongated channels, wherein each channel defines an axis and has a first end and a second end, and wherein each channel is juxtaposed in parallel with at least one other said channel;
a first section made of an electrophoretic filter medium positioned inside each said channel at the first end thereof; and
a second section made of an electrophoretic filter medium positioned inside each said channel at the second end thereof to create a well in said channel between said first section and said second section for receiving a lysate therein to confine impurities from the lysate in the well when an electric field is applied and subsequently reversed in a substantially axial direction through the channel.
